(19) United States
(12) Patent Application Publication
(10) Pub. No.: US 2020/0054563 A1
(43) Pub. Date: Feb. 20, 2020

(54) PHARMACEUTICAL COMPOSITION FOR IN VIVO DELIVERY, METHOD OF PREPARATION OF A SUBSTANTIALLY WATER-INSOLUBLE PHARMACOLOGICALLY ACTIVE AGENT FOR IN VIVO DELIVERY, AND METHOD OF TREATING DISEASE

(71) Applicant: ZY Therapeutics Inc., Cary, NC (US)

(72) Inventors: Jun Li, Cary, NC (US); Jian Bao, Cary, NC (US); Aili Rong, Cary, NC (US)

(73) Assignee: ZY Therapeutics Inc., Cary, NC (US)

(21) Appl. No.: 16/659,171

(22) Filed: Oct. 21, 2019

Related U.S. Application Data
(63) Continuation-in-part of application No. PCT/US2018/028900, filed on Apr. 23, 2018.
(60) Provisional application No. 62/489,198, filed on Apr. 24, 2017.

(51) Int. Cl. A61K 9/16; A61K 9/19; A61K 31/337

(52) U.S. Cl. CPC A61K 9/1682; A61K 31/337; A61K 9/19; A61K 9/1658

(57) ABSTRACT

The present application discloses a pharmaceutical composition for in vivo delivery. The pharmaceutical composition includes a pharmacologically active agent and a pharmaceutically acceptable carrier. The pharmaceutically acceptable carrier includes a biocompatible polymer. The biocompatible polymer and the pharmacologically active agent are formulated as particles. The pharmaceutical composition is free of a water-immiscible solvent.

### CROSS-REFERENCE TO RELATED APPLICATION

This application is a continuation-in-part of International Application No. PCT/US2018/028900, filed Apr. 23, 2018, which claims priority to U.S. Provisional Patent Application No. 62/489,198, filed Apr. 24, 2017, the contents of which are incorporated by reference in the entirety.

### TECHNICAL FIELD

The present invention relates to a pharmaceutical composition for in vivo delivery, a method of preparation of a substantially water-insoluble pharmacologically active agent for in vivo delivery, and a method of treating a disease using the pharmaceutical composition.

### BACKGROUND

Poor bioavailability of water insoluble compounds has long been a problem in the pharmaceutical and diagnostics industry. While compounds with an aqueous solubility of greater than 1% w/v are not expected to present dissolution-related bioavailability and absorption problems, many new chemical entities exhibit aqueous solubility much below this value. Many highly useful compounds are dropped from development or are formulated in a manner otherwise undesirable due to poor water solubility.

Albumin-based nanoparticle compositions have been developed as a drug delivery system for delivering substantially water insoluble drugs such as a taxane. See, for example, U.S. Pat. Nos. 5,916,596, 6,506,405, 6,749,868, 6,537,579, 7,820,788, and 7,923,536. Abraxane®, an albumin stabilized nanoparticle formulation of paclitaxel, was approved in the United States in 2005 and subsequently in various other countries for treating metastatic breast cancer. It was recently approved for treating non-small cell lung cancer in the United States, and has also shown therapeutic efficacy in various clinical trials for treating difficult-to-treat cancers such as pancreatic cancer and melanoma. Albumin derived from human blood has been used for the manufacture of Abraxane® as well as various other albumin-based nanoparticle compositions.

### SUMMARY

In one aspect, the present invention provides a method for preparation of a substantially water-insoluble pharmacologically active agent for in vivo delivery, comprising homogenizing a mixture comprising a pharmacologically active agent dispersed in a water-miscible solvent and a biocompatible polymer in an aqueous medium

Optionally, the mixture comprising the pharmacologically active agent dispersed the water-miscible solvent is substantially free of a water-immiscible solvent.

Optionally, the mixture comprising the pharmacologically active agent dispersed the water-miscible solvent is substantially free of a chlorinated solvent.

Optionally, the mixture comprising the pharmacologically active agent dispersed the water-miscible solvent is substantially free of chloroform and dichloromethane.

Optionally, homogenizing the mixture comprises subjecting the mixture to high shear conditions in a high pressure homogenizer at a pressure in a range of approximately 2,000 psi to approximately 30,000 psi.

Optionally, the high shear conditions comprises subjecting the mixture to the high shear conditions in in the high pressure homogenizer at a pressure in a range of approximately 10,000 psi to approximately 30,000 psi.

Optionally, homogenizing the mixture further comprises, prior to subjecting the mixture to the high shear conditions, subjecting the mixture to low shear conditions in a homogenizer operated in a range of approximately 100 rpm to approximately 28,000 rpm.

Optionally, the low shear conditions comprises subjecting the mixture to the low shear conditions in the homogenizer operated in a range of approximately 1,000 rpm to approximately 15,000 rpm.

Optionally, the method further comprises maintaining the mixture in a pH range suitable for stabilizing the pharmacologically active agent.

Optionally, the mixture is maintained at a pH in a range of approximately 4.0 to approximately 8.0.

Optionally, homogenizing the mixture produces particles comprising the pharmacologically active agent coated with the biocompatible polymer.

Optionally, the particles have an average diameter of less than 350 nm.

Optionally, subsequent to homogenizing the mixture, further comprising sterile filtering the mixture.

Optionally, subsequent to homogenizing the mixture, further comprising lyophilizing the mixture to obtain particles comprising the pharmacologically active agent coated with the biocompatible polymer.

Optionally, lyophilizing the mixture comprises lyophilizing the mixture in presence of an excipient.

Optionally, the excipient is a compound selected form a group consisting of sorbitol, sucrose, trehalose, mannitol, maltose, dextrose, lactose, glycerol, Dextran (70K), PVP (40K), Ficoll, gelatin, glycine, alanine, histidine, sodium citrate, sodium acetate, monosodium phosphate, sodium chloride.

Optionally, the pharmacologically active agent has a solubility in the water-miscible solvent of at least 0.5 mg/ml.

Optionally, the water-miscible solvent is a solvent selected from a group consisting of ethanol, propanol, butanol, acetone, acetonitrile, propylene glycol, PEG 300, PEG 400, glycerin, ethyl formate, dimethylacetamide(DMA), and N-Methyl-2-pyrrolidone(NMP).

Optionally, the water-miscible solvent is ethanol.

Optionally, the biocompatible polymer is albumin.

Optionally, the aqueous medium is selected from a group consisting of water, buffered aqueous media, saline, buffered saline, solutions of amino acids, solutions of sugars, solutions of vitamins, solutions of carbohydrates, and a combination of two or more thereof.

Optionally, the substantially water-insoluble pharmacologically active agent is selected from a group consisting of a pharmaceutically active agent, a diagnostic agent, and an agent of nutritional value.

Optionally, the pharmaceutically active agent is selected from a group consisting of analgesics/antipyretics, anesthetics, antiasthamatics, antibiotics, antidepressants, antidiabetics, antifungal agents, antihypertensive agents, anti-inflammatories, antineoplastics, antianxiety agents, immunosuppressive agents, antimigraine agents, sedatives/hypnotics, antianginal agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterial agents, antiviral agents, antimicrobials, anti-infectives, bronchodialators, hormones, hypoglycemic agents, hypolipidemic agents, proteins, nucleic acids, agents useful for erythropoiesis stimulation, antiulcer/antireflux agents, antinauseants/antiemetics, oil-soluble vitamins, as well as mitotane, visadine, halonitrosoureas, anthrocyclines and ellipticine.

Optionally, the pharmaceutically active agent is an antineoplastic selected from adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferon, camptothecin and derivatives thereof, phenesterine, paclitaxel and derivatives thereof, taxotere and derivatives thereof, vinblastine, vincristine, tamoxifen, etoposide or piposulfan.

Optionally, the pharmaceutically active agent is an immunosuppressive agent selected from cyclosporine, azathioprine, mizoribine or FK506 (tacrolimus).

Optionally, the diagnostic agent is selected from ultrasound contrast agents, radiocontrast agents, or magnetic contrast agents.

Optionally, the agent of nutritional value is selected from amino acids, sugars, proteins, carbohydrates, fat-soluble vitamins, or fat, or combinations of any two or more thereof.

Optionally, the biocompatible polymer is a naturally occurring polymer, a synthetic polymer, or a combination thereof.

Optionally, the naturally occurring polymer is selected from proteins, peptides, polynucleic acids, polysaccharides, proteoglycans or lipoproteins.

Optionally, the synthetic polymer is selected from synthetic polyamino acids containing cysteine residues and/or disulfide groups; polyvinyl alcohol modified to contain free sulfhydryl groups and/or disulfide groups; polyhydroxyethyl methacrylate modified to contain free sulfhydryl groups and/or disulfide groups; polyacrylic acid modified to contain free sulfhydryl groups and/or disulfide groups; polyethyloxazoline modified to contain free sulfhydryl groups and/or disulfide groups; polyacrylamide modified to contain free sulfhydryl groups and/or disulfide groups; polyvinyl pyrrolidinone modified to contain free sulfhydryl groups and/or disulfide groups; polyalkylene glycols modified to contain free sulfhydryl groups and/or disulfide groups; polylactides, polyglycolides, polycaprolactones, or copolymers thereof, modified to contain free sulfhydryl groups and/or disulfide groups; as well as mixtures of any two or more thereof.

In another aspect, the present invention provides a pharmaceutical composition for in vivo delivery comprising a pharmacologically active agent and a pharmaceutically acceptable carrier, the pharmaceutically acceptable carrier comprising a biocompatible polymer, the biocompatible polymer and the pharmacologically active agent being formulated as particles; wherein the pharmaceutical composition is free of a water-immiscible solvent.

Optionally, the pharmaceutical composition is for injection.

Optionally, the pharmaceutical composition is free of a chlorinated solvent.

Optionally, the pharmaceutical composition is free of chloroform and dichloromethane.

Optionally, the biocompatible polymer is albumin.

Optionally, the pharmacologically active agent is selected from a group consisting of a pharmaceutically active agent, a diagnostic agent, and an agent of nutritional value.

Optionally, the pharmaceutically active agent is selected from a group consisting of analgesics/antipyretics, anesthetics, antiasthamatics, antibiotics, antidepressants, antidiabetics, antifungal agents, antihypertensive agents, anti-inflammatories, antineoplastics, antianxiety agents, immunosuppressive agents, antimigraine agents, sedatives/hypnotics, antianginal agents, antipsychotic agents, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterial agents, antiviral agents, antimicrobials, anti-infectives, bronchodialators, hormones, hypoglycemic agents, hypolipidemic agents, proteins, nucleic acids, agents useful for erythropoiesis stimulation, antiulcer/antireflux agents, antinauseants/antiemetics, oil-soluble vitamins, as well as mitotane, visadine, halonitrosoureas, anthrocyclines and ellipticine.

Optionally, the pharmaceutically active agent is an antineoplastic selected from adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferon, camptothecin and derivatives thereof, phenesterine, paclitaxel and derivatives thereof, taxotere and derivatives thereof, vinblastine, vincristine, tamoxifen, etoposide or piposulfan.

Optionally, the pharmaceutically active agent is an immunosuppressive agent selected from cyclosporine, azathioprine, mizoribine or FK506 (tacrolimus).

Optionally, the diagnostic agent is selected from ultrasound contrast agents, radiocontrast agents, or magnetic contrast agents.

Optionally, the agent of nutritional value is selected from amino acids, sugars, proteins, carbohydrates, fat-soluble vitamins, or fat, or combinations of any two or more thereof.

Optionally, the biocompatible polymer is a naturally occurring polymer, a synthetic polymer, or a combination thereof.

Optionally, the naturally occurring polymer is selected from proteins, peptides, polynucleic acids, polysaccharides, proteoglycans or lipoproteins.

Optionally, the synthetic polymer is selected from synthetic polyamino acids containing cysteine residues and/or disulfide groups; polyvinyl alcohol modified to contain free sulfhydryl groups and/or disulfide groups; polyhydroxyethyl methacrylate modified to contain free sulfhydryl groups and/or disulfide groups; polyacrylic acid modified to contain free sulfhydryl groups and/or disulfide groups; polyethyloxazoline modified to contain free sulfhydryl groups and/or disulfide groups; polyacrylamide modified to contain free sulfhydryl groups and/or disulfide groups; polyvinyl pyrrolidinone modified to contain free sulfhydryl groups and/or disulfide groups; polyalkylene glycols modified to contain free sulfhydryl groups and/or disulfide groups; polylactides, polyglycolides, polycaprolactones, or copolymers thereof, modified to contain free sulfhydryl groups and/or disulfide groups; as well as mixtures of any two or more thereof.

Optionally, the pharmacologically active agent is paclitaxel, and the biocompatible polymer is albumin.

Optionally, a ratio (w/w) of albumin to the paclitaxel in the pharmaceutical composition is 1:1 to 9:1.

In another aspect, the present invention provides a method of treating a disease comprising administering an effective amount of a pharmaceutical composition described herein, wherein the disease is cancer, arthritis, or restenosis.

Optionally, the disease is cancer.

Optionally, the pharmaceutical composition is administered intravenously, intraarterially, intrapulmonarily, orally, by inhalation, intravesicularly, intramuscularly, intra-tracheally, subcutaneously, intraocularly, intrathecally, or transdermally.

Optionally, the pharmaceutical composition is administered intravenously.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows the dynamic light scattering (DLS) results of the nanosuspension prepared at pH 6.4 before filtration.

FIG. 1B shows the dynamic light scattering (DLS) results of the nanosuspension prepared at pH 6.4 after filtration.

FIG. 2A shows HPLC spectra of Paclitaxel in the standard solution.

FIG. 2B shows HPLC spectra of Paclitaxel in a formulation prepared by ethanol and pH change.

FIG. 2C shows HPLC spectra of Paclitaxel in a formulation prepared by ethanol without pH change. The split peak in HPLC may indicate the degradation of the drug during the process.

### DETAILED DESCRIPTION

Convention methods for formulating drug-containing nanoparticles typically includes dissolving a pharmacologically active agent in a water-immiscible solvent, dissolving a biocompatible polymer in an aqueous medium, homogenizing the pharmacologically active agent solution and the biocompatible polymer solution to obtain an emulsion, and evaporating the emulsion under vacuum. For example, nanoparticle albumin-bound (NAB) technology has been used to formulating paclitaxel-containing nanoparticles, i.e., Abraxane. NAB-technology, or other conventional drug-containing nanoparticles formulating methods, heavily relies on the use of organic solvent, particularly chlorinated solvents such as chloroform and dichloromethane. The residual chlorinated solvents in the nanoparticles introduce toxicity, which poses a potential risk to patient health. For example, the residual chloroform concentration in some batches of Abraxane formulations may be as high as 118 ppm to 2962 ppm (“Assessment report for Abraxane, European Medicines Agency, Doc. Ref.: EMEA/47053/2008). A commonly acceptable limit of chloroform in pharmaceutical products is 60 ppm.

Many attempts were made to mitigate the toxicities of the toxic solvent-based formulating methods, but without success. U.S. Pat. Nos. 5,916,596 and 6,749,868 described the use of water-miscible solvent alone for formulating the paclitaxel-containing nanoparticles, and concluded that the water-miscible solvent alone is “[n]ot suitable for invention process” (see, e.g., Examples 11 and 12 of U.S. Pat No. 5,916,596 and Examples 12 and 13 of U.S. Pat. No. 6,749,868). The inventors of U.S. Pat. Nos. 5,916,596 and 6,749,868 discovered that the use of water-miscible solvent alone failed to produce nano-particles, and the particles generated by their experiments “were too large for intravenous injection” and have “very broad particle size distribution.” The inventors of US Patent Nos. 5,916,596 and 6,749,868 concluded that the NAB manufacturing method “requires . . . water immiscible solvents to enable formation of . . . nanoparticles.” Because drug-containing nanoparticles prepared using organic solvents in NAB manufacturing would inherently contain residual organic solvents, this may lead to chronic toxicities even if the residual organic solvent is below the commonly acceptable limit.

Accordingly, the present disclosure provides, inter alia, a pharmaceutical composition for in vivo delivery, a method for preparation of a substantially water-insoluble pharmacologically active agent for in vivo delivery, and a method of treating a disease that substantially obviate one or more of the problems due to limitations and disadvantages of the related art. In one aspect, the present disclosure provides a method for preparation of a substantially water-insoluble pharmacologically active agent for in vivo delivery. In some embodiments, the method includes homogenizing a mixture comprising the pharmacologically active agent dispersed a water-miscible solvent and a biocompatible polymer in an aqueous medium. Contrary to the teaching in the prior art, the present inventors have surprisingly discovered that pharmaceutical compositions formulated as nanoparticles for in vivo delivery can be prepared using water-miscible solvents alone. In the present method, the mixture for preparing the pharmaceutical composition is substantially free of a water-immiscible solvent such as a chlorinated solvent (e.g., chloroform and dichloromethane). As a result, the pharmaceutical composition for in vivo delivery prepared by the present method is free of any residual water-immiscible solvent, obviating the issue of toxicity introduced by the water-immiscible solvent.

As used herein, the term “water miscible solvent” refers to a solvent which forms a one phase, homogenous solution when combined with water. Optionally, a water miscible solvent is a solvent that, at 20 Celsius degrees, can be mixed with water without phase separation at a concentration of at least 2% v/v, e.g., at least 5% v/v, at least 10% v/v, at least 20% v/v, and at least 50% v/v.

As used herein, the term “in vivo delivery” refers to delivery of a pharmacologically active agent by a variety of routes of administration, as are well known to those of skill in the art. Thus, exemplary routes of administration include topical, oral, intraarticular, intracisternal, intraocular, intraventricular, intrathecal, intravenous, intramuscular, intraperitoneal, intradermal/transdermal/subcutaneous, intratracheal/inhalational, rectal (i.e., via suppository), vaginal (i.e., via pessary), intracranial, intraurethral, intrahepatic, intraarterial, intratumoral, mucosal, and the like, as well as suitable combinations of any two or more thereof. Further, administration of the pharmacologically active agent contemplated for use in the present invention can be systemic (i.e., administered to the subject as a whole via any of the above routes) or localized (i.e., administered to the specific location of the particular infirmity of the subject via any of the above routes).

As used herein, the term “biocompatible” refers to a substance that does not appreciably alter or affect in any adverse way, the biological system into which it is introduced.

In some embodiments, the method includes preparing a first solution in which the pharmacologically active agent is dispersed a water-miscible solvent. Optionally, the pharmacologically active agent is dissolved in the water-miscible solvent. Optionally, the first solution is a supersaturated solution of the pharmacologically active agent in the water-miscible solvent. Optionally, the first solution is a saturated solution of the pharmacologically active agent in the water-miscible solvent. Optionally, the first solution is an under-saturated solution of the pharmacologically active agent in the water-miscible solvent. Optionally, the first solution is a transparent solution which contains no suspension of undissolved pharmacologically active agent particles. Optionally, the first solution further includes water.

Various appropriate water-miscible solvents may be used for preparing the first solution having the pharmacologically active agent is dispersed the water-miscible solvent. Examples of appropriate water-miscible solvents include, but are not limited to, water miscible alcohols (e.g., methanol, ethanol, isopropyl alcohol, n-propanol, n-butanol, isobutanol, sec-butanol, tert-butanol, methoxyethanol, ethoxyethanol, 3-methyl-1-butanol, 1-pentanol), water-miscible diols such as propylene glycol, water-miscible polyols such as polyethylene glycol (e.g., polyethylene glycol 300, polyethylene glycol 400), organic acids (e.g., acetic acid, formic acid, trichloroacetic acid, trifluoroacetic acid), acetone, acetonitrile, ethyl formate, dimethylacetamide(DMA), N-Methyl-2-pyrrolidone (NMP), tetrahydrofuran, 1,4-dioxane, dimethyl sulfoxide, dimethylformamide, dimethylacetamide, methyltetrahydrofuran, and a combination of two or more thereof.

Optionally, the water-miscible solvent is a water-miscible solvent in which the pharmacologically active agent has a solubility of at least 0.5 mg/ml, e.g., at least 1.0 mg/ml, at least 1.5 mg/ml, at least 2 mg/ml, at least 5 mg/ml, at least 7.5 mg/ml, at least 10 mg/ml, at least 15 mg/ml, or at least 20 mg/ml.

Unlike conventional methods for nanoparticle formulation, the first solution in the present method is substantially free of a water-immiscible solvent. Optionally, the first solution is substantially free of a chlorinated solvent such as chloroform and dichloromethane.

In some embodiments, the method further includes preparing a second solution in which the biocompatible polymer is dissolved in an aqueous medium. Examples of appropriate aqueous medium include, but are not limited to, water, buffered aqueous media, saline, buffered saline, solutions of amino acids, solutions of sugars, solutions of vitamins, solutions of carbohydrates, and a combination of two or more thereof. The second solution in the present method is substantially free of a water-immiscible solvent. Optionally, the second solution is substantially free of a chlorinated solvent such as chloroform and dichloromethane.

In some embodiments, the method further includes homogenizing a mixture of the first solution and the second solution as prepared by the method described herein. Optionally, the mixture is homogenized to form an emulsion.

In some embodiments, the step of homogenizing the mixture includes subjecting the mixture to high shear conditions. Various appropriate high-shear homogenization methods may be used for homogenizing the mixture. Examples of appropriate homogenization methods include, but are not limited to, high pressure homogenization, high shear mixers, sonication, high shear impellers, and the like. In some embodiments, the high-shear homogenizing step is performed using a high pressure homogenizer. Optionally, the homogenizing step is performed using a high pressure homogenizer at a pressure in a range of approximately 2,000 psi to approximately 30,000 psi, e.g., approximately 10,000 psi to approximately 30,000 psi, approximately 20,000 psi to approximately 30,000 psi, or approximately 25,000 psi to approximately 30,000 psi. The resulting emulsion includes nanodroplets of the dissolved pharmacologically active agent and nanodroplets of dissolved biocompatible polymer.

In some embodiments, the step of homogenizing the mixture further includes, prior to subjecting the mixture to the high shear conditions, subjecting the mixture to low shear conditions. Various appropriate low-shear homogenization methods may be used for homogenizing the mixture. Examples of appropriate homogenizers include, but are not limited to, a conventional laboratory homogenizer and a magnetic stirrer mixer. In some embodiments, the low-shear homogenizing step is performed using a homogenizer operated in a range of approximately 100 rpm to approximately 28,000 rpm, e.g., approximately 1,000 rpm to approximately 15,000 rpm, approximately 3,000 rpm to approximately 10,000 rpm, or approximately 5,000 rpm to approximately 15,000 rpm.

In some embodiments, the method further includes maintaining the mixture in a pH range suitable for stabilizing the pharmacologically active agent. For example, the method may include preparing the second solution or the first solution using a buffer so that the pH of the mixture of the first solution and the second solution during the step of homogenization may be maintained in a range suitable for stabilizing the pharmacologically active agent. In another example, the method may include adjusting the pH of the mixture prior to or during the step of homogenization so that the pH of the mixture may be maintained in a range suitable for stabilizing the pharmacologically active agent. In yet another example, the method further includes measuring the pH of the mixture, and if the pH of the mixture is outside the range suitable for stabilizing the pharmacologically active agent, adjusting the pH of the mixture prior to or during the step of homogenization so that the pH of the mixture may be maintained in a range suitable for stabilizing the pharmacologically active agent. Optionally, the method includes maintaining the mixture in a pH range of 4.0 to 8.0, e.g., 4.0 to 4.5, 4.5 to 5.0, 5.0 to 5.5, 5.5 to 6.0, 6.0 to 6.5, 6.5 to 7.0, 7.0 to 7.5, or 7.5 to 8.0.

Different pharmacologically active agents may require different pH ranges to avoid degradation during the homogenization process. As further detailed in the Examples, paclitaxel may undergo degradation during the homogenization process when the pH of the mixture is maintained at a pH range of approximately 7.0 to approximately 7.4, (e.g., approximately 7.2). Degradation of paclitaxel may be avoided if the pH of the mixture is maintained in a range of approximately 5.0 to approximately 6.5, e.g., approximately 5.0 to approximately 5.5, approximately 5.5 to approximately 6.5, and approximately 6.0 to approximately 6.5.

In some embodiments, homogenizing the mixture produces particles including the pharmacologically active agent coated with the biocompatible polymer. Optionally, the particles produced by the present method have an average diameter of less than 1 micron, e.g., less than 450 nm, less than 400 nm, less than 350 nm, less than 300 nm, less than 250 nm, less than 220 nm, less than 200 nm, less than 180 nm. Optionally, the particles produced by the present method have an average diameter in a range of approximately 10 nm to approximately 220 nm, e.g., approximately 10 nm to approximately 200 nm, approximately 50 nm to approximately 180 nm, and approximately 50 nm to approximately 170 nm. Such particles are capable of being sterile-filtered before use in the form of a liquid suspension.

In some embodiments, subsequent to homogenizing the mixture, the method further includes, sterile filtering the homogenized mixture. Optionally, the solution having the particles including the pharmacologically active agent coated with the biocompatible polymer is sterile filtered through a 0.22 micron filter.

In some embodiments, the method further includes lyophilizing the homogenized mixture to obtain particles including the pharmacologically active agent coated with the biocompatible polymer. Optionally, the step of lyophilizing the homogenized mixture is performed subsequent to the step of sterile filtering the homogenized mixture. Optionally, the method further includes adding an excipient to the homogenized mixture prior to the step of lyophilizing the homogenized mixture, and lyophilizing the homogenized mixture in the presence of an excipient. Various appropriate excipients may be used, including sorbitol, sucrose, trehalose, mannitol, maltose, dextrose, lactose, glycerol, Dextran (70K), PVP (40K), Ficoll, gelatin, glycine, alanine, histidine, sodium citrate, sodium acetate, monosodium phosphate, sodium chloride, or a combination of two or more thereof.

In some embodiments, the pharmacologically active agent is a substantially water-insoluble pharmacologically active agent. The present method can be applied to substantially water-insoluble pharmacologically active agent without pre-modifying the substantially water-insoluble pharmacologically active agent to enhance water solubility of the substantially water-insoluble pharmacologically active agent. For example, the present method can be applied to substantially water-insoluble pharmacologically active agent without pegylating the substantially water-insoluble pharmacologically active agent to enhance the solubility of the substantially water-insoluble pharmacologically active agent.

In some embodiments, the pharmacologically active agent is an anti-neoplastic agent (e.g., an anti-cancer drug). Examples of anti-neoplastic agents include alkylating agents, antimetabolites, natural anticancer products, hormones, metal coordination complexes and mixtures thereof. Optionally, the anti-neoplastic agent is paclitaxel, decotaxel, or doxorubicin, or derivatives or analogues thereof, or any combination thereof.

In some embodiments, the pharmacologically active agent is a taxane. Examples of taxanes include paclitaxel, docetaxel, cabazitaxel, larotaxel, ortataxel, tesetaxel, 10-deacetyl analogues of paclitaxel, and derivatives and analogs thereof.

In some embodiments, the pharmacologically active agent is a cytotoxic agent. Examples of cytotoxic agents include alkylating agents (e.g., chlorambucil, cyclophosphamide, melphalan, cyclopropane), anthracycline antitumor antibiotics (e.g., doxorubicin, daunomycin, adriamycin, mitomycin C, 2-(hydroxymethyl)anthraquinone), antimetabolites (e.g., methotrexate, dichloromethatrexate), cisplatin, carboplatin, metallopeptides containing platinum, copper, vanadium, iron, cobalt, gold, cadmium, zinc and nickel, deoxynivalenol, thymidine, pentamethylmelamin, dianhydrogalactitol, 5-Methyl-THF, anguidine, maytansine, neocarzinostatin, chlorozotocin, AZQ, 2′-deoxycoformycin, PALA, valrubicin, m-AMSA and misonidazole.

In some embodiments, the pharmacologically active agent is a hydrophobic drug. Examples of hydrophobic drugs include glucocorticoids, cytostatics, certain antibodies, drugs acting on immunophilins, interferons, opiates, INF binding proteins, mycophenolate, FTY720, cyclosporin (including cyclosporin A, cyclosporin B, cyclosporin C, cyclosporin D, cyclosporin E, cyclosporin F, cyclosporin G, cyclosporin H, cyclosporin I), tacrolimus (FK506, PROGRAF®), sirolimus (rapamycin, RAPAMUNE®), everolimus (RAD, CERTICAN®), taxanes such as paclitaxel, discodermolide, colchicine, vinca alkaloids such as vinblastine or vincristine, and analogues or derivatives of any of the listed agents

In some embodiments, the pharmacologically active agent is selected from a group consisting of a pharmaceutically active agent, a diagnostic agent, and an agent of nutritional value. Examples of pharmaceutically active agents further include:

analgesics/antipyretics (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and the like);

anesthetics (e.g., cyclopropane, enflurane, halothane, isoflurane, methoxyflurane, nitrous oxide, propofol, and the like);

antiasthamatics (e.g., Azelastine, Ketotifen, Traxanox, and the like);

antibiotics (e.g., neomycin, streptomycin, chloramphenicol, cephalosporin, ampicillin, penicillin, tetracycline, and the like);

antidepressants (e.g., nefopam, oxypertine, doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, desipramine hydrochloride, trimipramine maleate, protriptyline hydrochloride, and the like);

antidiabetics (e.g., biguanides, hormones, sulfonylurea derivatives, and the like);

antifungal agents (e.g., griseofulvin, keloconazole, amphotericin B, Nystatin, candicidin, and the like);

antihypertensive agents (e.g., propanolol, propafenone, oxyprenolol, Nifedipine, reserpine, trimethaphan camsylate, phenoxybenzamine hydrochloride, pargyline hydrochloride, deserpidine, diazoxide, guanethidine monosulfate, minoxidil, rescinnamine, sodium nitroprusside, rauwolfia serpentina, alseroxylon, phentolamine mesylate, reserpine, and the like);

anti-inflammatories (e.g., (non-steroidal) indomethacin, naproxen, ibuprofen, ramifenazone, piroxicam, (steroidal) cortisone, dexamethasone, fluazacort, hydrocortisone, prednisolone, prednisone, and the like);

antineoplastics (e.g., adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, and the like);

antianxiety agents (e.g., lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, dantrolene, and the like);

immunosuppressive agents (e.g., cyclosporine, azathioprine, mizoribine, FK506 (tacrolimus), and the like); antimigraine agents (e.g., ergotamine tartrate, propanolol hydrochloride, isometheptene mucate, dichloralphenazone, and the like);

sedatives/hypnotics (e.g., barbiturates (e.g., pentobarbital, pentobarbital sodium, secobarbital sodium), benzodiazapines (e.g., flurazepam hydrochloride, triazolam, tomazeparm, midazolam hydrochloride, and the like);

antianginal agents (e.g., beta-adrenergic blockers, calcium channel blockers (e.g., nifedipine, diltiazem hydrochloride, and the like), nitrates (e.g., nitroglycerin, isosorbide dinitrate, pentaerythritol tetranitrate, erythrityl tetranitrate, and the like));

antipsychotic agents (e.g., haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, prochlorperazine, and the like);

antimanic agents (e.g., lithium carbonate);

antiarrhythmics (e.g., bretylium tosylate, esmolol hydrochloride, verapamil hydrochloride, amiodarone, encainide hydrochloride, digoxin, digitoxin, mexiletine hydrochloride, disopyramide phosphate, procainamide hydrochloride, quinidine sulfate, quinidine gluconate, quinidine polygalacturonate, flecainide acetate, tocainide hydrochloride, lidocaine hydrochloride, and the like);

antiarthritic agents (e.g., phenylbutazone, sulindac, penicillamine, salsalate, piroxicam, azathioprine, indomethacin, meclofenamate sodium, gold sodium thiomalate, ketoprofen, auranofin, aurothioglucose, tolmetin sodium, and the like);

antigout agents (e.g., colchicine, allopurinol, and the like);

anticoagulants (e.g., heparin, heparin sodium, warfarin sodium, and the like);

thrombolytic agents (e.g., urokinase, streptokinase, altoplase, and the like);

antifibrinolytic agents (e.g., aminocaproic acid);

hemorheologic agents (e.g., pentoxifylline);

antiplatelet agents (e.g., aspirin, empirin, ascriptin, and the like);

anticonvulsants (e.g., valproic acid, divalproate sodium, phenytoin, phenytoin sodium, clonazepam, primidone, phenobarbitol, phenobarbitol sodium, carbamazepine, amobarbital sodium, methsuximide, metharbital, mephobarbital, mephenytoin, phensuximide, paramethadione, ethotoin, phenacemide, secobarbitol sodium, clorazepate dipotassium, trimethadione, and the like);

antiparkinson agents (e.g., ethosuximide, and the like);

antihistamines/antipruritics (e.g., hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, trimprazine tartrate and the like);

agents useful for calcium regulation (e.g., calcitonin, parathyroid hormone, and the like);

antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, colistin sulfate, and the like);

antiviral agents (e.g., interferon gamma, zidovudine, amantadine hydrochloride, ribavirin, acyclovir, and the like);

antimicrobials (e.g., cephalosporins (e.g., cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, and the like), penicillins (e.g., ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G potassium, penicillin G procaine, methicillin sodium, nafcillin sodium, and the like), erythromycins (e.g., erythromycin ethylsuccinate, erythromycin, erythromycin estolate, erythromycin lactobionate, erythromycin siearate, erythromycin ethylsuccinate, and the like), tetracyclines (e.g., tetracycline hydrochloride, doxycycline hyclate, minocycline hydrochloride, and the like), and the like);

anti-infectives (e.g., GM-CSF);

bronchodialators (e.g., sympathomimetics (e.g., epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate), anticholinergic agents (e.g., ipratropium bromide), xanthines (e.g., aminophylline, dyphylline, metaproterenol sulfate, aminophylline), mast cell stabilizers (e.g., cromolyn sodium), inhalant corticosteroids (e.g., flurisolidebeclomethasone dipropionate, beclomethasone dipropionate monohydrate), salbutamol, beclomethasone dipropionate (BDP), ipratropium bromide, budesonide, ketotifen, salmeterol, xinafoate, terbutaline sulfate, triamcinolone, theophylline, nedocromil sodium, metaproterenol sulfate, albuterol, flunisolide, and the like);

hormones (e.g., androgens (e.g., danazol, testosterone cypionate, fluoxymesterone, ethyltostosterone, testosterone enanihate, methyltestosterone, fluoxymesterone, testosterone cypionate), estrogens (e.g., estradiol, estropipate, conjugated estrogens), progestins (e.g., methoxyprogesterone acetate, norethindrone acetate), corticosteroids (e.g., triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate, and the like), thyroid hormones (e.g., levothyroxine sodium) and the like), and the like;

hypoglycemic agents (e.g., human insulin, purified beef insulin, purified pork insulin, glyburide, chlorpropamide, glipizide, tolbutamide, tolazamide, and the like);

hypolipidemic agents (e.g., clofibrate, dextrothyroxine sodium, probucol, lovastatin, niacin, and the like);

proteins (e.g., DNase, alginase, superoxide dismutase, lipase, and the like);

nucleic acids (e.g., sense or anti-sense nucleic acids encoding any therapeutically useful protein, including any of the proteins described herein, and the like);

agents useful for erythropoiesis stimulation (e.g., erythropoietin);

antiulcer/antireflux agents (e.g., famotidine, cimetidine, ranitidine hydrochloride, and the like);

antinauseants/antiemetics (e.g., meclizine hydrochloride, nabilone, prochlorperazine, dimenhydrinate, promethazine hydrochloride, thiethylperazine, scopolamine, and the like);

oil-soluble vitamins (e.g., vitamins A, D, E, K, and the like);

as well as other drugs such as mitotane, visadine, halonitrosoureas, anthrocyclines, ellipticine, and the like.

Examples of diagnostic agents contemplated for use in the practice of the present disclosure include ultrasound contrast agents, radiocontrast agents (e.g., iodo-octanes, halocarbons, renografin, and the like), magnetic contrast agents (e.g., fluorocarbons, lipid soluble paramagnetic compounds, and the like), as well as other diagnostic agents which cannot readily be delivered without some physical and/or chemical modification to accommodate the substantially water insoluble nature thereof.

Examples of agents of nutritional value contemplated for use in the practice of the present disclosure include amino acids, sugars, proteins, carbohydrates, fat-soluble vitamins (e.g., vitamins A, D, E, K, and the like) or fat, or combinations of any two or more thereof.

Optionally, the pharmaceutically active agent is paclitaxel.

In some embodiments, the biocompatible polymer is a naturally occurring polymer, a synthetic polymer, or a combination thereof. Examples of naturally occurring polymers include, but are not limited to, proteins, peptides, polynucleic acids, polysaccharides (e.g., starch, cellulose, dextrans, alginates, chitosan, pectin, hyaluronic acid, and the like), proteoglycans, and lipoproteins. Examples of proteins for use as stabilizing agents in accordance with the present disclosure include, but are not limited to, albumins, immunoglobulins, caseins, insulins, hemoglobins, lysozymes, immunoglobulins, α-2-macroglobulin, fibronectins, vitronectins, fibrinogens, lipases, and the like. Proteins, peptides, enzymes, antibodies and combinations thereof, are general classes of stabilizers contemplated for use in the present disclosure. Optionally, the biocompatible polymer is albumin, e.g., human serum albumin.

Examples of synthetic polymers for use as stabilizing agents in accordance with the present disclosure include, but are not limited to, synthetic polyamino acids containing cysteine residues and/or disulfide groups; polyvinyl alcohol modified to contain free sulfhydryl groups and/or disulfide groups; polyhydroxyethyl methacrylate modified to contain free sulfhydryl groups and/or disulfide groups; polyacrylic acid modified to contain free sulfhydryl groups and/or disulfide groups; polyethyloxazoline modified to contain free sulfhydryl groups and/or disulfide groups; polyacrylamide modified to contain free sulfhydryl groups and/or disulfide groups; polyvinyl pyrrolidinone modified to contain free sulfhydryl groups and/or disulfide groups; polyalkylene glycols modified to contain free sulfhydryl groups and/or disulfide groups; polylactides, polyglycolides, polycaprolactones, or copolymers thereof, modified to contain free sulfhydryl groups and/or disulfide groups; as well as mixtures of any two or more thereof.

In another aspect, the present disclosure provides a pharmaceutical composition for in vivo delivery including a pharmacologically active agent and a pharmaceutically acceptable carrier. In some embodiments, the pharmaceutically acceptable carrier includes a biocompatible polymer, the biocompatible polymer and the pharmacologically active agent being formulated as particles, e.g., nanoparticles. Optionally, the pharmaceutical composition is for injection.

The present pharmaceutical composition for in vivo delivery is free of any detectable water-immiscible solvent. Optionally, the pharmaceutical composition for in vivo delivery is free of any chlorinated solvent. Optionally, the pharmaceutical composition for in vivo delivery is free of chloroform and dichloromethane. Optionally, the pharmaceutical composition for in vivo delivery is also free of Cremophor.

In some embodiments, the pharmaceutical composition is a nanoparticle protein-bound drug composition. Optionally, the pharmaceutical composition is a nanoparticle protein-bound cancer drug composition. Optionally, the pharmaceutical composition is a nanoparticle protein-bound taxane drug composition. Optionally, the pharmaceutically active agent is a taxane and the biocompatible polymer is protein.

In some embodiments, the pharmaceutical composition is a nanoparticle albumin-bound drug composition. Optionally, the pharmaceutical composition is a nanoparticle albumin-bound cancer drug composition. Optionally, the pharmaceutical composition is a nanoparticle albumin-bound taxane drug composition. Optionally, the pharmaceutically active agent is a taxane and the biocompatible polymer is albumin, e.g., human serum albumin.

Optionally, the pharmaceutically active agent is paclitaxel. Optionally, the biocompatible polymer is albumin, e.g., human serum albumin. Optionally, the pharmaceutically active agent is paclitaxel, and the biocompatible polymer is albumin. Optionally, a ratio (w/w) of albumin to the paclitaxel in the pharmaceutical composition is 1:1 to 9:1. Optionally, a ratio (w/w) of albumin to the paclitaxel in the pharmaceutical composition is 1:1 to 5:1. Optionally, a ratio (w/w) of albumin to the paclitaxel in the pharmaceutical composition is approximately 9:1.

In another aspect, the present disclosure provides a method of treating a disease. In some embodiments, the method includes administering an effective amount of a pharmaceutical composition described herein. Optionally, the disease is cancer, arthritis, or restenosis. Optionally, the cancer is breast cancer, ovarian cancer, lung cancer, colon cancer, pancreatic cancer, endometrial cancer, chronic leukemia, sarcoma, ovarian carcinoma, rectal cancer, throat cancer, melanoma, bladder cancer, kidney cancer, mammary adenocarcinoma, gastrointestinal cancer, stomach cancer, prostate cancer, or Kaposi's sarcoma.

Optionally, the pharmaceutical composition is administered intravenously, intraarterially, intrapulmonarily, orally, by inhalation, intravesicularly, intramuscularly, intra-tracheally, subcutaneously, intraocularly, intrathecally, or transdermally. Optionally, the pharmaceutical composition is administered via topical, enteral/gastrointestinal, parenteral, epidural, intracerebral, intracerebroventrical, intradermal, subcutaneous, nasal, intraosseous infusion, intravitreal, intravesical, or transmucosal route.

Exemplary means for the systemic administration of pharmacologically active agent(s) are well known to those of skill in the art, and include oral (for example, with a sustained release formulation of the pharmacologically active agent), continuous IV infusion, infusion via bolus injection, infusion through in-dwelling catheters, and any other means which can function to deliver the pharmacologically active agent systemically to the patient in need thereof, and the like, and suitable combinations of any two or more thereof.

Exemplary means for the localized administration of pharmacologically active agent(s) include catheters, implantable or portable infusion devices, slow release delivery vehicles, and any other means which can function to deliver the pharmacologically active agent to the localized area of the infirmity to be treated, and the like, and suitable combinations of any two or more thereof.

Implantable or portable infusion devices contemplated for use in the present invention are well known to those of skill in the art, and include devices which can deliver precise and controlled amounts of the pharmacologically active agent over extended periods. Typically, these are driven by electromagnetic force, and/or osmotic force, and/or hydrostatic force, and/or gaseous pressure, and/or mechanical force. Commonly, implantable infusion devices are capable of being periodically refilled, and of being able to receive the pharmacologically active agent in solid or liquid form.

Exemplary slow release delivery vehicles include, for example, pharmacologically active agent(s) encapsulated in a colloidal dispersion system or in a polymer stabilized system. Useful colloidal dispersion systems include nanocapsules, microspheres, beads, lipid-based systems (including oil-in-water emulsions, micelles, mixed micelles, liposomes, and the like), and the like. The colloidal system presently preferred is a liposome or microsphere. Liposomes are artificial membrane vesicles which are useful as slow release delivery vehicles when injected or implanted. Some examples of lipid-polymer conjugates and liposomes are disclosed in U.S. Pat. No., 5,631,018, which is incorporated herein by reference in its entirety. Other examples of slow release delivery vehicles are biodegradable hydrogel matrices (U.S. Pat. No, 5,041,292), dendritic polymer conjugates (U.S. Pat. No. 5,714,166), and multivesicular liposomes (Depofoam®, Depotech, San Diego, Calif.) (U.S. Pat. Nos. 5,723,147 and 5,766,627). One type of microspheres suitable for encapsulating therapeutic agents for local injection (e.g., into subdermal tissue) is poly(D,L)lactide microspheres, as described in D. Fletcher, Anesth. Analg. 84:90-94, 1997.

Besides delivering an effective therapeutic dose to the site of the infirmity and decreasing the chance of systemic toxicity, localized administration also decreases the exposure of the pharmacologically active agent to degradative processes, such as proteolytic degradation and immunological intervention via antigenic and immunogenic responses, as well as to systemic clearance processes, such as sequestration in the liver,

It is to be understood that clinical applications of the present disclosure are not limited to cancers or neoplastic diseases or conditions. Rather, any disease or condition may benefit from the use of the microparticles and compositions disclosed herein, provided there are suitable agents that can be entrapped in the microparticle, suitable routes for administration, suitable target population of subjects, and suitable methods to monitor a subject's response to the agent(s) to be delivered using the microparticle. In particular examples, the diseases or conditions that can benefit from the use of the microparticles and compositions include and are not limited to viral infections, e.g., HIV infection or AIDS, or HBV or HCV infections; autoimmune diseases, e.g., lupus or rheumatoid arthritis; neurodegenerative diseases, e.g., Parkinson's disease or Alzheimer's disease.

The following examples are intended to further describe and illustrate various aspects of the disclosure, but not to limit, the scope of the disclosure in any manner, shape, or form, either explicitly or implicitly.

### EXAMPLE 1

### Preparation of Nanosuspension by a High Pressure Homogenizer with High Yield at pH 6.4

287 mg of Human Serum Albumin (HSA) was dissolved in 28.7 ml DI Water to make a clear solution. The polymer solution was filtered through a 0.22 μm membrane and the pH value of the filtrate was measured to be 7.2. 20.0 μl of acetic acid (1.0 M) solution was added in the HSA solution to adjust the pH to around 6.4. 30 mg of Paclitaxel was dissolved in 1.3 ml of ethanol. The HSA and the Paclitaxel solution were premixed by a homogenous dispersing machine (AngNi Instruments, Model AD500S-H) at 8,000 rpm for 1 minute. The mixture was poured into a high shear homogenizer (Microfluidics Inc, MA, model LM-20) and homogenized for 10 minutes at a pressure of 30,000 psi. The resulting dispersion was translucent with an average particle size of 110 nm and a polydispersity index (PDI) of 0.133. (analyzed using a Malvern Zetasizer instrument).

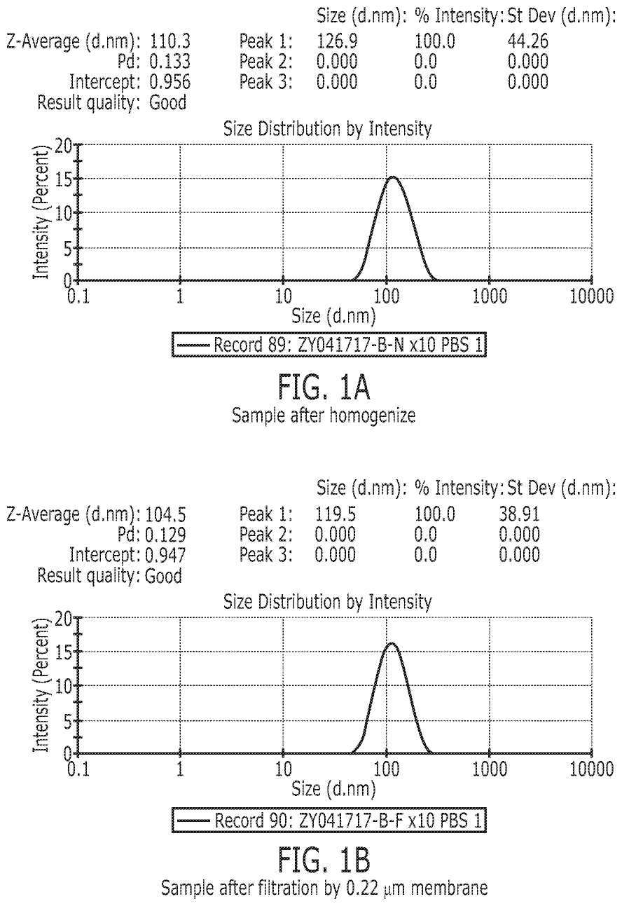

The formulation was filtered through 0.22 μm membrane for sterilization. A typical dynamic light scattering (DLS) result was shown in FIG. 1A and FIG. 1B. FIG. 1A shows the dynamic light scattering (DLS) results of the nanosuspension prepared at pH 6.4 before filtration. FIG. 1B shows the dynamic light scattering (DLS) results of the nanosuspension prepared at pH 6.4 after filtration. The Paclitaxel content before (FIG. 1A) and after (FIG. 1B) filtration was measured by HPLC. The results in Table 1 showed that the yield was almost 100% and there was no loss of the product during the filtration step.

TABLE 1
Paclitaxel concentration in the nanosuspension before and after
filtration when the pH of the HSA solution was kept at 6.4.

| | | | |
|---|---|---|---|
| | Peak Area RT | | PTX concentration |
| | 8.6 min | PTX Concentration | in undiluted sample |
| Dilution | (mAu*s) | (ug/mL) | (ug/mL) |

041717-B-F

| | | | |
|---|---|---|---|
| 10x | 1956.10 | 43.64 | 436.36 |

041717-B-N

| | | | |
|---|---|---|---|
| 10x | 1910.30 | 42.60 | 426.00 |

| | |
|---|---|
| PTX recovery % after the filtration | 102.43 |

### EXAMPLE 2

### Preparation of Nanosuspension by a High Pressure Homogenizer with High Yield at pH 5.8

287 mg of Human Serum Albumin (HSA) was dissolved in 28.7 ml DI Water to make a clear solution. The polymer solution was filtered through a 0.22 μm membrane and the pH value of the filtrate was measured to be 7.2. 30.0 μl of acetic acid (1.0 M) aqueous solution was added into the HSA solution to adjust the pH to around 5.8. 30 mg of Paclitaxel was dissolved in 1.3 ml of ethanol. The HSA and the Paclitaxel solution were premixed by a homogenous dispersing machine (AngNi Instruments, Model AD500S-H) at 8,000 rpm for 1 minute. The mixture was poured into a high shear homogenizer (Microfluidics Inc, MA, model LM-20) and homogenized for 10 minutes at a pressure of 30,000 psi. The resulting dispersion was translucent with an average particle size of 130 nm and a PDI of 0.114 (analyzed using a Malvern Zetasizer instrument).

The formulation was filtered through a 0.22 μm membrane for sterilization. The Paclitaxel content before and after filtration was measured by HPLC. The results showed that recovery of 95% of the drug product during the filtration step.

### EXAMPLE 3

### Preparation of Nanosuspension by a High Pressure Homogenizer with Low Yield at pH 5.5

287 mg of Human Serum Albumin (HSA) was dissolved in 28.7 ml DI Water to make a clear solution. The polymer solution was filtered through a 0.22 μm membrane and the pH value of the filtrate was measured to be 7.2. 40.0 μl of acetic acid (1.0 M) solution was added in the HSA solution to adjust the pH to around 5.5. 30 mg of Paclitaxel was dissolved in 1.3 ml of ethanol. The HSA and the Paclitaxel solution were premixed by a homogenous dispersing machine (AngNi Instruments, Model AD500S-H) at 8,000 rpm for 1 minute. The mixture was poured into a high shear homogenizer (Microfluidics Inc, MA, model LM-20) and homogenized for 10 minutes at a pressure of 30,000 psi. The resulting dispersion was translucent with an average particle size of 154 nm and a PDI of 0.102 (analyzed using a Malvern Zetasizer instrument).

The formulation was filtered through a 0.22 μm membrane for sterilization. The Paclitaxel content before and after filtration was measured by HPLC. The results in Table 2 showed that the recovery yield is 68% during the filtration step. Therefore, the pH of the HSA solution is a critical factor to obtain the high yield during filtration step.

TABLE 2
Paclitaxel concentration in the nanosuspension before and after
filtration when the pH of the HSA solution was kept at 5.5.

| | | | |
|---|---|---|---|
| | Peak Area RT | | PTX concentration |
| | 7.9 min | PTX Concentration | in undiluted sample |
| Dilution | (mAu*s) | (ug/mL) | (ug/mL) |

041717-A-F

| | | | |
|---|---|---|---|
| 10x | 1386.80 | 30.76 | 307.59 |

041717-A-N

| | | | |
|---|---|---|---|
| 10x | 2001.80 | 44.67 | 446.70 |

| | |
|---|---|
| PTX recovery % after the filtration | 68.86 |

### EXAMPLE 4

### Instability of Nanosuspension prepared by a High Pressure Homogenizer at pH 4.8

287 mg of Human Serum Albumin (HSA) was dissolved in 28.7 ml DI Water to make a clear solution. The polymer solution was filtered through a 0.22 μm membrane and the pH value of the filtrate was measured to be 7.2. 80.0 μl of acetic acid (1.0 M) solution was added in the HSA solution to adjust the pH to around 4.8. 30 mg of Paclitaxel was dissolved in 1.3 ml of ethanol. The HSA and the Paclitaxel solution were premixed by a homogenous dispersing machine (AngNi Instruments, Model AD500S-H) at 8,000 rpm for 1 minute. The mixture was poured into a high shear homogenizer (Microfluidics Inc, MA, model LM-20) and homogenized for 10 minutes at a pressure of 30,000 psi. The resulting dispersion was translucent with an average particle size of 127 nm and a PDI of 0.138 (analyzed using a Malvern Zetasizer instrument).

The formulation was filtered through a 0.22 μm membrane for sterilization. The Paclitaxel content before and after filtration was measured by HPLC. The results showed that the recovery yield is 64% during the filtration step. Meanwhile, it is observed that the formulation yields precipitation, suggesting particle aggregation after 4 hours even at a reduced temperature of 4 ° C.

### EXAMPLE 5

### Preparation of Nanosuspension by a High Pressure Homogenizer with High Yield for Filtration Step at pH 4.0 or Lower

287 mg of Human Serum Albumin (HSA) was dissolved in 28.7 ml DI Water to make a clear solution. The polymer solution was filtered through a 0.22 μm membrane and the pH value of the filtrate was measured to be 7.2. 65.0 μl of acetic acid (5.0 M) solution was added in the HSA solution to adjust the pH to around 4.0. 30 mg of Paclitaxel was dissolved in 1.3 ml of ethanol. The HSA and the Paclitaxel solution were premixed by a homogenous dispersing machine (AngNi Instruments, Model AD500S-H) at 8,000 rpm for 1 minute. The mixture was poured into a high shear homogenizer (Microfluidics Inc, MA, model LM-20) and homogenized for 10 minutes at a pressure of 30,000 psi. The resulting dispersion was translucent with an average particle size of 128 nm and a PDI of 0.103 (Malvern Zetasizer).

The formulation was filtered through a 0.22 μm membrane for sterilization. The Paclitaxel content before and after filtration was measured by HPLC. The results showed that the yield is 86% during the filtration step when the pH was 4.0 or lower.

### EXAMPLE 6

### Preparation of a Lyophilized Dosage Form of the Nanosuspension Prepared by a High Pressure Homogenizer at pH 7.2, and Subsequent Reconstitution and Stability Study

360 mg of Human Serum Albumin (HSA) was dissolve in 30 ml DI Water to make a clear solution. The polymer solution was filtered through a 0.22 μm membrane and the pH value of the filtrate was measured to be 7.2. 40 mg of Paclitaxel was dissolved in 1.0 ml of ethanol. The HSA and the Paclitaxel solution were premixed by a homogenous dispersing machine (AngNi Instruments, Model AD500S-H) at 8,000 rpm for 1 minute. The mixture was poured into a high shear homogenizer (Microfluidics Inc, MA, model LM-20) and homogenized for 10 minutes at a pressure of 30,000 psi. The resulting dispersion was translucent with an average particle size of 111 nm and a PDI of 0.112 (analyzed using a Malvern Zetasizer instrument).

The formulation was filtered through a 0.22 μm membrane for sterilization resulting in a nanosuspension with Zeta-average at 106 nm (PDI=0.086). The Paclitaxel content before and after filtration was measured by HPLC. The results showed that the yield was 92% during the filtration step.

To obtain a lyophilized dosage form, 5% of Mannitol, Sucrose, Trehalose or Sorbitol were respectively added into different samples of dispersion solution followed by freeze-drying for at least 48 hrs. The resulting cakes were white powders.

To reconstitute the suspension, DI water was added to the lyophilized powder, and the mixture was shaken mildly till it formed a homogenous suspension. The reconstituted suspension was then tested as following. The particle size before and after reconstitution are listed in Table 3. Without excipients, the reconstituted solution was milky, easy to aggregate even stored at 4 degree, and shown with a relatively large particle size. With excipients such as sucrose, sorbitol and trehalose, the cakes were easily reconstituted to the original suspension, and the final formulation could be stored at 4° C. without any significant change of particle size for at least 2 hour.

TABLE 3
The particle size of the nanosuspension before and after lyophilization in the presence
of different excipients. Short term stability study (up to 2 h) was investigated at 4° C.

| | Before Lyo | After Lyo | | Store 1 hr at 4° C. | | Store 2 hrs at 4° C. | |
|---|---|---|---|---|---|---|---|
| Formulation | (Size nm/PDI) | (Size nm/PDI) | | (Size nm/PDI) | | (Size nm/PDI) | |
| No Excipient | 106 nm (0.086) | 175.0 | (0.517) | Large aggregate | | N/A | |
| 5% Sorbitol | | 114 nm | (0.114) | 112 nm | (0.102) | 113 nm | (0.090) |
| 5% Surcose | | 113 nm | (0.085) | 112 | (0.112) | 112 | (0.122) |
| 5% Trehalose | | 119 nm | (0.114) | 115 nm | (0.137) | 119 nm | (0.122) |

| | | | | |
|---|---|---|---|---|
| 5% Mannitol | 3823 | (0.389) | Large aggregate | N/A |

### EXAMPLE 7

### Unstable Drug Substance in the Nanosuspension Prepared by High Pressure Homogenizer at pH 7.2 or Higher

287 mg of Human Serum Albumin (HSA) was dissolve in 28.7 ml DI Water to make a clear solution. The polymer solution was filtered through a 0.22 μm membrane and the pH value of the filtrate was measured to be 7.2. 30 mg of Paclitaxel was dissolved in 1.3 ml of ethanol. The HSA and the Paclitaxel solution were premixed by a homogenous dispersing machine (AngNi Instruments, Model AD500S-H) at 8,000 rpm for 1 minute. The mixture was poured into a high shear homogenizer (Microfluidics Inc, MA, model LM-20) and homogenized for 10 minutes at a pressure of 30,000 psi. The resulting dispersion was translucent with an average particle size of 123 nm and a PDI of 0.118 (analyzed using a Malvern Zetasizer instrument).

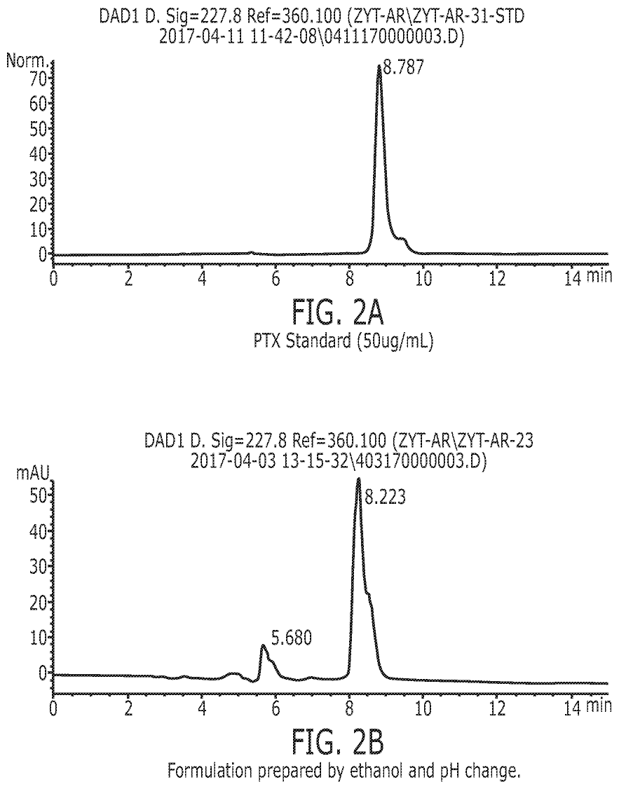

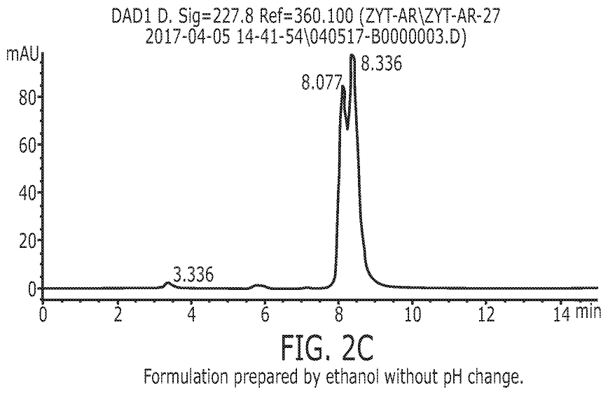

The formulation was filtered through a 0.22 μm membrane for sterilization. The Paclitaxel content before and after filtration was measured by HPLC. FIG. 2A shows HPLC spectra of Paclitaxel in the standard solution. FIG. 2B shows HPLC spectra of Paclitaxel in a formulation prepared by ethanol and pH change. FIG. 2C shows HPLC spectra of Paclitaxel in a formulation prepared by ethanol without pH change. The split peak in HPLC may indicate the degradation of the drug during the process. The results showed that the recovery yield was 95% during the filtration step (FIG. 2B). However, it was observed in some cases that the drug substance (Paclitaxel) was not stable during the process, resulting additional peak in HPLC spectra (FIG. 2C).

All patent filings, other publications, accession numbers and the like cited above are incorporated by reference in their entirety for all purposes to the same extent as if each individual item were specifically and individually indicated to be so incorporated by reference. If different variants of a sequence are associated with an accession number at different times, the version associated with the accession number at the filing date of this application is meant. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

1. A method for preparation of a substantially water-insoluble pharmacologically active agent for in vivo delivery, comprising:
homogenizing a mixture comprising a pharmacologically active agent dispersed in a water-miscible solvent and a biocompatible polymer in an aqueous medium;
wherein the mixture comprising the pharmacologically active agent dispersed the water-miscible solvent is substantially free of a water-immiscible solvent.

2. (canceled)

3. The method of claim 1, wherein the mixture comprising the pharmacologically active agent dispersed the water-miscible solvent is substantially free of a chlorinated solvent.

4. The method of claim 1, wherein the mixture comprising the pharmacologically active agent dispersed the water-miscible solvent is substantially free of chloroform and dichloromethane.

5. The method of claim 1, wherein homogenizing the mixture comprises subjecting the mixture to high shear conditions in a high pressure homogenizer at a pressure in a range of approximately 2,000 psi to approximately 30,000 psi.

6. The method of claim 5, wherein the high shear conditions comprises subjecting the mixture to the high shear conditions in in the high pressure homogenizer at a pressure in a range of approximately 10,000 psi to approximately 30,000 psi.

7. The method of claim 5, homogenizing the mixture further comprises, prior to subjecting the mixture to the high shear conditions, subjecting the mixture to low shear conditions in a homogenizer operated in a range of approximately 100 rpm to approximately 28,000 rpm.

8. The method of claim 7, wherein the low shear conditions comprises subjecting the mixture to the low shear conditions in the homogenizer operated in a range of approximately 1,000 rpm to approximately 15,000 rpm.

9. The method of claim 1, further comprising maintaining the mixture in a pH range suitable for stabilizing the pharmacologically active agent.

10. The method of claim 9, wherein the mixture is maintained at a pH in a range of approximately 4.0 to approximately 8.0.

11. The method of claim 1, wherein homogenizing the mixture produces particles comprising the pharmacologically active agent coated with the biocompatible polymer.

12. The method of claim 11, wherein the particles have an average diameter of less than 350 nm.

13. The method of claim 12, subsequent to homogenizing the mixture, further comprising sterile filtering the mixture.

14. The method of claim 1, subsequent to homogenizing the mixture, further comprising lyophilizing the mixture to obtain particles comprising the pharmacologically active agent coated with the biocompatible polymer.

15. The method of claim 14, wherein lyophilizing the mixture comprises lyophilizing the mixture in presence of an excipient.

16. The method of claim 15, wherein the excipient is a compound selected form a group consisting of sorbitol, sucrose, trehalose, mannitol, maltose, dextrose, lactose, glycerol, Dextran (70K), PVP (40K), Ficoll, gelatin, glycine, alanine, histidine, sodium citrate, sodium acetate, monosodium phosphate, sodium chloride.

17. The method of claim 1, wherein the pharmacologically active agent has a solubility in the water-miscible solvent of at least 0.5 mg/ml.

18. The method of claim 1, wherein the water-miscible solvent is a solvent selected from a group consisting of ethanol, propanol, butanol, acetone, acetonitrile, propylene glycol, PEG 300, PEG 400, glycerin, ethyl formate, dimethylacetamide(DMA), and N-Methyl-2-pyrrolidone(NMP).

19. The method of claim 17, wherein the water-miscible solvent is ethanol.

20. The method of claim 1, wherein the biocompatible polymer is albumin.

21-30. (canceled)

31. A pharmaceutical composition for in vivo delivery comprising a pharmacologically active agent and a pharmaceutically acceptable carrier, the pharmaceutically acceptable carrier comprising a biocompatible polymer, the biocompatible polymer and the pharmacologically active agent being formulated as particles;
wherein the pharmaceutical composition is free of a water-immiscible solvent.

32-50. (canceled)
